(19) United States
(12) Patent Application Publication
(10) Pub. No.: US 2026/0114803 A1
(43) Pub. Date: Apr. 30, 2026

(54) EVALUATION APPARATUS AND EVALUATION SYSTEM

(71) Applicant: PARAMOUNT BED CO., LTD., Tokyo (JP)

(72) Inventors: Saori TOYOTA, Tokyo (JP); Takamasa KOGURE, Tokyo (JP)

(21) Appl. No.: 19/208,801

(22) Filed: May 15, 2025

(30) Foreign Application Priority Data
Oct. 25, 2024 (JP) 2024-188636

(51) Int. Cl. A61B 5/00; A61B 5/11

(52) U.S. Cl. CPC A61B 5/4821; A61B 5/1118

(57) ABSTRACT

An evaluation apparatus includes: a biosignal acquisition unit configured to acquire a biosignal of a patient; and a controller. The controller is configured to calculate an amount of activity of the patient based on the biosignal acquired from the biosignal acquisition unit, and evaluate a sedative scale (Richmond Agitation-Sedation Scale (RASS)) of the patient based on the calculated amount of activity.

### FIELD

Embodiments discussed herein relate to an evaluation apparatus and the like. This application is based on Japanese Patent Application No. 2024-188636, filed in Japan on Oct. 25, 2024, the contents of which are incorporated into this application.

### BACKGROUND

The inventions in which whether an abnormality occurs in a user is determined from a biological information value and the like of a patient have been known.

As a scale for evaluating a sedative state of a patient in use of a sedative drug, an evaluation based on Richmond Agitation-Sedation Scale (RASS) is used in an intensive care unit, palliative care, and the like.

### BRIEF DESCRIPTION OF THE DRAWINGS

FIG. 1 is a diagram illustrating the whole in a first embodiment.

FIG. 2 is a diagram illustrating a functional configuration of hardware in the first embodiment.

FIG. 3A is a diagram illustrating a functional configuration of software in the first embodiment, and FIG. 3B is a diagram illustrating one example of a determination threshold table.

FIG. 4 is a diagram illustrating RASS in the first embodiment.

FIG. 5 is an operation flow illustrating processing in the first embodiment.

FIG. 6 is a diagram illustrating an example in the first embodiment.

FIG. 7 is an operation flow illustrating processing in a second embodiment.

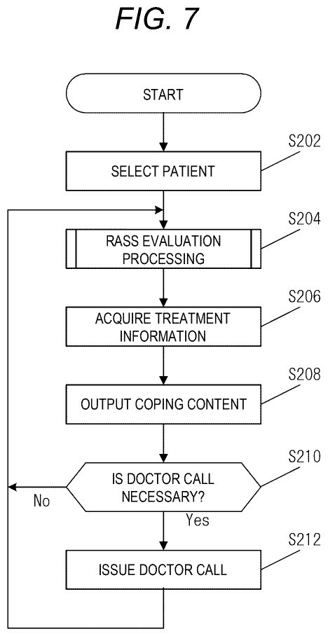

FIG. 8 is a diagram illustrating an example in a third embodiment.

FIGS. 9A and 9B are diagrams illustrating an example in a fourth embodiment.

FIG. 10 is an operation flow illustrating processing in a fifth embodiment.

### DETAILED DESCRIPTION

One or more embodiments are now described with reference to the drawings, wherein like reference numerals are used to refer to like elements throughout. In the following description, for purposes of explanation, numerous specific details are set forth in order to provide a thorough understanding of the various embodiments. It is evident, however, that the various embodiments can be practiced without these specific details (and without applying to any particular networked environment or standard).

As used in this disclosure, in some embodiments, the terms “component,” “system” and the like are intended to refer to, or comprise, a computer-related entity or an entity related to an operational apparatus with one or more specific functionalities, wherein the entity can be either hardware, or a combination of hardware and software in execution.

One or more components may reside within a process and/or thread of execution and a component may be localized on one computer and/or distributed between two or more computers. In addition, these components can execute from various computer readable media having various data structures stored thereon. The components may communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software application or firmware application executed by a processor, wherein the processor can be internal or external to the apparatus and executes at least a part of the software or firmware application. As yet another example, a component can be an apparatus that provides specific functionality through electronic components without mechanical parts, the electronic components can comprise a processor therein to execute software stored on a non-transitory electronic memory or firmware that confers at least in part the functionality of the electronic components. While various components have been illustrated as separate components, it will be appreciated that multiple components can be implemented as a single component, or a single component can be implemented as multiple components, without departing from example embodiments. Further, the various embodiments can be implemented as a method, apparatus or article of manufacture using standard programming and/or engineering techniques to produce software, firmware, hardware or any combination thereof to control a computer to implement the disclosed subject matter. The term “article of manufacture” as used herein is intended to encompass a computer-readable (or machine-readable) device or computer-readable (or machine-readable) storage/communications media having a computer program stored thereon. For example, computer readable storage media can comprise, but are not limited to, magnetic storage devices (e.g., hard disk, floppy disk, magnetic strips), optical disks (e.g., compact disk (CD), digital versatile disk (DVD)), smart cards, and flash memory devices (e.g., card, stick, key drive). Of course, those skilled in the art will recognize many modifications can be made to this configuration without departing from the scope or spirit of the various embodiments.

In addition, the words “example” and “exemplary” are used herein to mean serving as an instance or illustration. Any embodiment or design described herein as “example” or “exemplary” is not necessarily to be construed as preferred or advantageous over other embodiments or designs. Rather, use of the word example or exemplary is intended to present concepts in a concrete fashion. As used in this application, the term “or” is intended to mean an inclusive “or” rather than an exclusive “or”. That is, unless specified otherwise or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

Embodiments described herein can be exploited in substantially any wireless communication technology, comprising, but not limited to, wireless fidelity (Wi-Fi), global system for mobile communications (GSM), universal mobile telecommunications system (UMTS), worldwide interoperability for microwave access (WiMAX), enhanced general packet radio service (enhanced GPRS), third generation partnership project (3GPP) long term evolution (LTE), third generation partnership project 2 (3GPP2) ultra mobile broadband (UMB), high speed packet access (HSPA), Z-Wave, Zigbee and other 802.XX wireless technologies and/or legacy telecommunication technologies.

In general, one aspect of the present application is an evaluation apparatus including: a biosignal acquisition unit configured to acquire a biosignal of a patient; and a controller. The controller is configured to calculate an amount of activity of the patient based on the biosignal acquired from the biosignal acquisition unit, and evaluate a sedative scale (Richmond Agitation-Sedation Scale (RASS)) of the patient based on the calculated amount of activity.

Another aspect of the present application is an evaluation system including: a sheet-shaped detection device that is placed under a patient, the detection device being configured to detect a body movement of the patient; a calculation device configured to calculate an amount of activity of the patient from the body movement; and an evaluation apparatus configured to evaluate a sedative scale (Richmond Agitation-Sedation Scale (RASS)) of the patient based on the amount of activity.

A description will hereinafter be made on one embodiment for implementing the present disclosure with reference to the drawings. Specifically, a case where an evaluation apparatus according to the present disclosure is applied will be described, however, the range to which the present disclosure is applied is not limited to the embodiment.

Richmond Agitation-Sedation Scale (RASS) is used as a scale for evaluating a sedative state of a patient in use of a sedative drug and a restlessness risk such as delirium. The RASS is an evaluation method of sedative levels that is positively used in clinical sites such as intensive care units and palliative care, and makes an evaluation of ten stages (−4 to +5) with 0 as the center. Staffs and others are expected to evaluate whether a purpose of a sedative drug used to a patient is appropriately attained at regular intervals (for example, intervals of one hour to several hours).

The staffs and others need to evaluate a score of a patient by observing and calling the patient during 30 seconds, for example, so that there has been such a problem that the evaluation takes labor and time. When a staff has evaluated that a patient is in a sedative state, the staff needs to evaluate the sedative state by providing call stimulation or body stimulation to the patient, so that there has been such a problem that this stimulation causes burden also for the patient.

The evaluation of the RASS is dependent on individual skills, so that there has also been such a problem that the evaluation varies depending on an individual difference and an experience difference among the staffs and others who make the evaluation.

In order to solve such problems, an evaluation apparatus capable of evaluating a sedative scale based on the amount of activity of a patient with an easy and simple method will be described by using the following embodiments. In the following embodiments, as a preferred example, a case where the evaluation apparatus is applied to RASS for palliative care will be described.

### 1. FIRST EMBODIMENT

### 1.1 Entire System

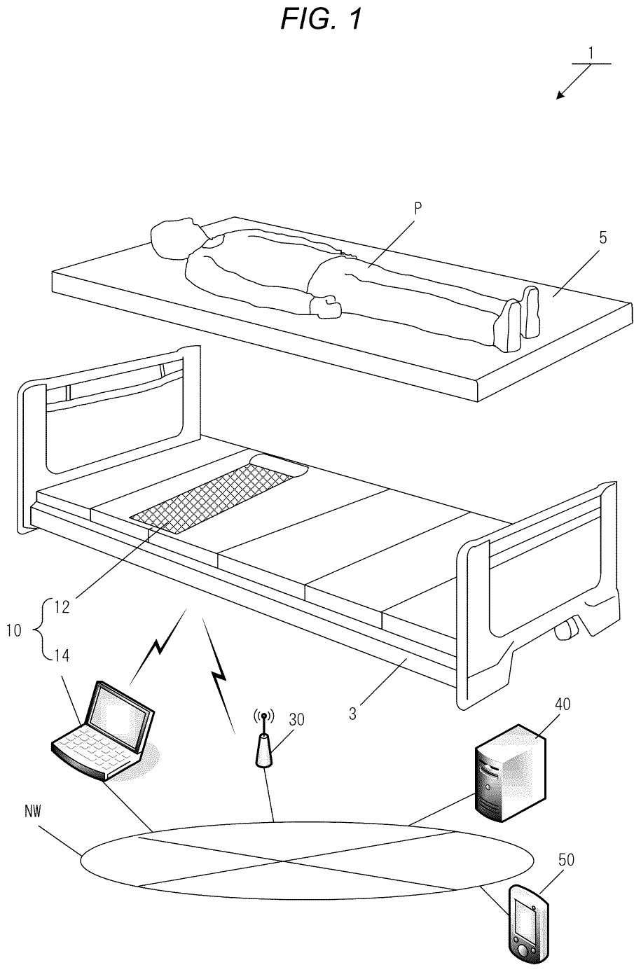

FIG. 1 is a diagram illustrating the whole overview of a system 1 to which an evaluation apparatus according to the present disclosure is applied. As illustrated in FIG. 1, the system 1 includes, for example, an evaluation apparatus 10 that evaluates a sedative scale (RASS) of a patient P. The evaluation apparatus 10 may be provided with a detection device 12 that is placed between sections of a bed 3 and a mattress 5, and a processor 14 for processing a value output from the detection device 12. The evaluation apparatus 10 may include, for example, the detection device 12 standalone having the function of the processor 14. Processor 14 may also function as a calculation device.

When a patient (hereinafter, referred to as “patient P” as one example) serving as a user whose sedative state is evaluated is present on the mattress 5, the detection device 12 detects a body vibration (vibration emitted from a human body) as a biosignal from the patient P. A biological information value of the patient P is then calculated based on the detected vibration. In the present embodiment, the detection device 12 may output and display the calculated biological information value as a biological information value of the patient P. The detection device 12 calculates at least an amount of activity of the patient P, and may calculate a heartbeat rate and a respiratory rate in addition.

The processor 14 may be a general device, and thus is not limited to an information processor such as a computer, but may include a device, for example, a tablet or a smartphone.

As a patient, a main target is a person whose sedative scale is evaluated, and is preferably a user to which a sedative drug has been administered, but the patient is not necessarily limited to the person in the state. For example, as a state of a patient, a person whose sedative scale needs to be managed by medication or the like, or a patient with a risk of occurring delirium may serve as targets.

The detection device 12 herein is formed in a sheet shape so as to have a thin thickness. This allows the detection device 12 even to be used without giving a discomfort feeling to the patient P when the detection device 12 is placed between the bed 3 and the mattress 5, so that biological information values including the amount of activity in the bed portion can be measured for long periods. In other words, biological information values and the like are acquired as a state of a patient when the patient is in bed rest and at rest.

The detection device 12 only needs to acquire biosignals (a body movement, a respiratory motion, a ballistocardiographic motion, and the like) of the patient P. In the present embodiment, the respiratory rate, the heartbeat rate, the amount of activity, and the like are calculated based on the body vibration, but may be detected using an infrared sensor, for example, a biosignal of the patient P may be acquired from the acquired video and the like, or an actuator with a distortion gage may be used. The detection device 12 may be implemented as a smartphone, a tablet, or the like that is placed on the bed 3 (or the mattress 5), for example, by using the incorporated acceleration sensor or the like.

The bed 3 is installed in a variety of places. For example, although the bed 3 is generally installed in a hospital where the patient P is hospitalized or a facility where the patient P occupies, the bed 3 may be installed in his/her house in home care or the like.

The evaluation apparatus 10 is communicable with another device via a network NW. The detection device 12 in the evaluation apparatus 10 may be connected to the network NW via the processor 14, for example, or the detection device 12 may be directly connected to the network NW via an access point 30 through a wireless LAN or the like. The detection device 12 may be directly connected to the network NW, for example, by a communication module communicable with mobile communication networks (LTE/4G/5G/6G and the like) being incorporated therein.

The network NW is connectable with, for example, a server device 40 and a terminal device 50. The server device 40 may store, for example, a biological information value and the like acquired by the evaluation apparatus 10, or may store the sedative scale evaluated by the evaluation apparatus 10. The server device 40 may be, for example, an electronic medical record server that stores disease information on a user.

For example, the terminal device 50 may be an information processor, such as a smartphone, a tablet, and a laptop computer, that is used by a medical staff such as a medical doctor and a nurse. The terminal device 50 may be an information processor that is used by a staff in the facility, a family, and other persons. The terminal device 50 may be an information processor that is used by a patient himself/herself for a self-check.

### 1.2 Functional Configuration

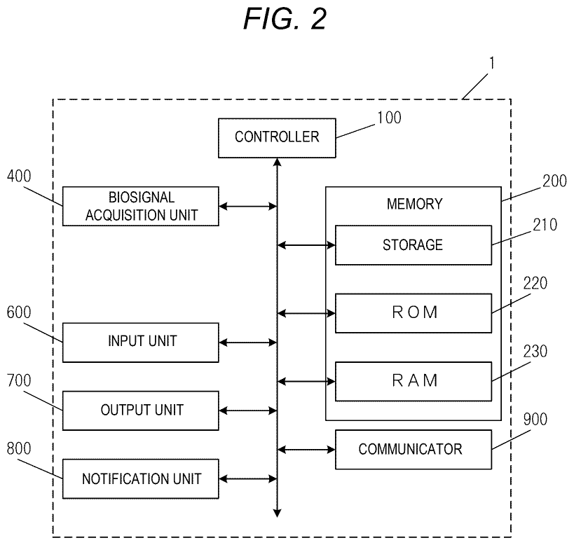

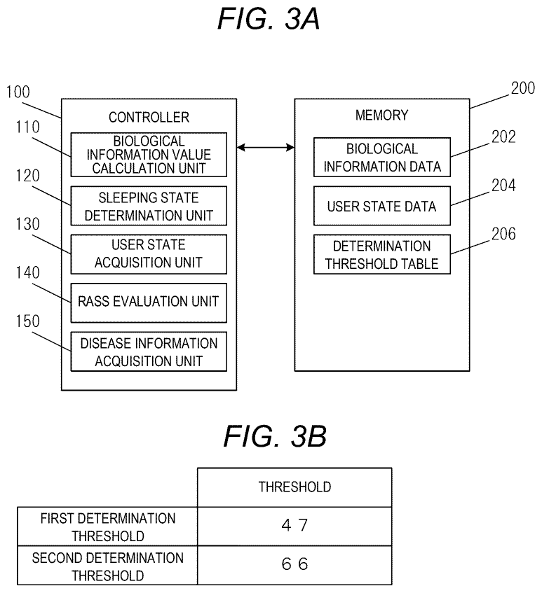

Next, a functional configuration of the evaluation apparatus 10 in the system 1 will be described using FIGS. 2 and 3. The evaluation apparatus 10 in the present embodiment includes the detection device 12 and the processor 14, and the respective functional units (processes) other than a biosignal acquisition unit 400 may be implemented by either of them. These devices are combined to function as the evaluation apparatus 10.

The evaluation apparatus 10 may perform a report (notification) operation in accordance with a sedative scale of a patient. In this time, a report destination may be a staff, a patient himself/herself, or a family. As a reporting method, a report (notification) may be made simply by means of sounds or a screen display, or may be made to the terminal device by means of an email or the like. A report (notice) may be made to another terminal device or the like.

### 1.2.1 Hardware Configuration

As illustrated in FIG. 2, the evaluation apparatus 10 includes a controller 100, a memory 200 (a storage 210, a ROM 220, and a RAM 230), the biosignal acquisition unit 400, an input unit 600, an output unit 700, a notification unit 800, and a communicator 900, the number of each unit being one or plural as necessary.

In the case of FIG. 1, the detection device 12 is provided with the controller 100, the biosignal acquisition unit 400, and the memory 200, and the processor 14 may be provided with the other units.

The controller 100 controls the whole of the evaluation apparatus 10. The controller 100 implements various functions by reading and executing various programs stored in the memory 200 (for example, the storage 210 or the ROM 220) serving as a storage device. The controller 100 may be implemented by one or a plurality of controllers/calculation devices (a central processing unit (CPU) and a system on a chip (SoC)). The controller 100 may include a control circuit.

The memory 200 stores data on various information. The memory 200 is generally a device including one or a plurality of the storages 210, the ROMs 220, and the RAMs 230, and stores data in any of which as necessary.

The storage 210 is a nonvolatile storage device that can store the programs and data. For example, the storage 210 may include a storage device such as a hard disk drive (HDD) and a solid state drive (SSD). The storage 210 may be an externally connectable USB memory or memory card. The storage 210 may be, for example, a storage area on a cloud.

The ROM 220 is a nonvolatile memory capable of keeping the program and the data even when power is turned off.

The RAM 230 is a main memory that is mainly used by the controller 100 when executing the processing. The RAM 230 is a rewritable memory that temporarily keeps the programs read from the storage 210 and the ROM 220 and the data including results at the time of execution.

The biosignal acquisition unit 400 acquires a biosignal of the patient P. In the present embodiment, as one example, a sensor that detects a change in pressure is used to acquire a body vibration that is one type of a biosignal. The controller 100 then converts the acquired body vibration into biological information value data such as a respiratory rate, a heartbeat rate, and an amount of activity, and outputs the converted biological information value data. In addition, based on the body vibration data acquired by the biosignal acquisition unit 400, the controller 100 can acquire a bed rest state (for example, whether the patient P is in a bed rest, bed presence, bed departure, edge sitting position, or the like) of the user, and also can acquire a sleeping state (sleeping, waking-up), as is described later.

The biosignal acquisition unit 400 in the present embodiment acquires a body vibration of a user by a pressure sensor, for example, and acquires breathing and heartbeat from the body vibration, but may acquire a biosignal by a load sensor from a change in the center of gravity position (body movement) of the user, may acquire a biosignal by a radar based on a displacement in a body surface or bedclothes, or may provide a microphone to acquire a biosignal based on sounds picked up by the microphone. The biosignal acquisition unit 400 only needs to acquire a biosignal of a user using any of the sensors.

In other words, the biosignal acquisition unit 400 may be connected with a device such as the detection device 12, or may receive a biosignal from an external device.

With the input unit 600, a measurement person inputs various conditions, and performs a manipulation input of a measurement start. The input unit 600 may be implemented by, for example, any of input units, such as a hardware key and a software key.

The output unit 700 is a functional unit that outputs information based on a sedative scale, and a biological information value, such as a sleeping state, a heartbeat rate, and a respiratory rate, and makes a notification of an abnormality. The output unit 700 may be a display device, such as a display, or may be a notification device (sound output device) that makes a notification of a warning or the like. The output unit 700 may be an external storage device that stores data, or a transmission device or the like that transmits data through a communication path. The output unit 700 may be a communication device when a report is made to another device.

The input unit 600 and the output unit 700 may be implemented by other devices. For example, the input unit 600 and the output unit 700 may be implemented by using a terminal device (for example, a smartphone or a tablet that is used by the user) connected via the communicator 900. In this time, the terminal device may be able to execute a program with which the controller 100, which is described later, implements the processing.

The notification unit 800 makes a notification to a user or the like. For example, the notification unit 800 may be a speaker that outputs a sound, an LED serving as a light emitting device, or the like. The notification unit 800 may make a notification to another device (for example, a terminal device, such as a smartphone of a patient, a nurse call, or the like).

The communicator 900 performs communication with another device. For example, the communicator 900 provides communication with a device in a short distance by a scheme, such as a wireless LAN (or a wired LAN), and Bluetooth (registered trademark). The communicator 900 may be a device that provides near field communication, such as NFC. The communicator 900 may provide communication by a scheme that allows mobile communication, such as 4G/LTE/5G/6G. The communicator 900 may be an interface (for example, a USB) or the like for performing communication with another device.

### 1.2.2 Software Configuration

With reference to FIG. 3A, a configuration of software will be described. For example, the controller 100 implements the respective functions by executing programs and applications stored in the memory 200 (for example, the storage 210, the ROM 220, and the RAM 230).

A biological information value calculation unit 110 calculates, for example, an amount of activity, as a biological information value of the patient P. The biological information value calculation unit 110 may detect a body vibration per sampling unit time from the biosignal acquisition unit 400, and calculate an amount of activity based on the number of times of the detected body vibrations. The biological information value calculation unit 110 may calculate an amount of activity from a change and a motion of a sleeping posture of a user.

Specifically, the biological information value calculation unit 110 counts a section in which body movement larger than the breathing and the heartbeat is present at the resolution of 16 Hz (per second 16 times) of the output value by the sensor, for example. In this process, the biological information value calculation unit 110 indicates the number of counts for one minute (count/min) as an amount of activity, and the maximum value is 960 (count/min).

The biological information value calculation unit 110 may calculate, as an amount of activity, the number of seconds during when body movement occurs per one minute by dividing the number of counts for one minute by 16. For example, 47 (count/min) that is a threshold at which a low RASS group is determined is divided by 16 to obtain 2.9 seconds.

In the present embodiment, the biological information value calculation unit 110 may extract a respiratory component and a heartbeat component from the body movement acquired by the biosignal acquisition unit 400, and obtain a respiratory rate and a heartbeat rate based on the breathing interval and the heartbeat interval. The biological information value calculation unit 110 may analyze (Fourier transform and the like) the periodicity of the body movement, and calculate a respiratory rate and a heartbeat rate from the peak frequency.

A sleeping state determination unit 120 determines a sleeping state of a user. For example, the sleeping state determination unit 120 determines a sleeping state of a user based on the biosignal acquired by the biosignal acquisition unit 400. The sleeping state determination unit 120 may determine two states of “waking-up” and “sleeping” as sleeping states. The sleeping state determination unit 120 may further determine the “sleeping” state as “REM sleep” or “non-REM sleep”, and further make a determination of multiple levels (sleeping depths) as the “sleeping” state.

The sleeping state determination unit 120 may determine the sleeping state and the waking-up state based on the magnitude of the amount of activity and the state of a time-series change in the amount of activity. For example, the sleeping state determination unit 120 does not need to determine the waking-up state if a temporal body movement is present. The sleeping state determination unit 120 may determine the waking-up state in a case where the body movement of the patient P continues to some extent.

A user state acquisition unit 130 acquires a state of a user. A state of a user is a general state related to the user, and for example, a load sensor or the like provided to the bed 3 is used to acquire a state of whether the user is in a bed departure or bed presence state, for example. The user state acquisition unit 130 may further acquire, when the user is in a bed presence state, a sleeping posture of the user and a sleeping position of the user. The user state acquisition unit 130 may acquire a state of the user based on the biosignal acquired in the biosignal acquisition unit 400, for example, as mentioned above, in addition to the load sensor or the like. The state of the user may include a state of whether the user is sleeping or waking-up based on the sleeping state of the user determined in the sleeping state determination unit 120. The user state acquisition unit 130 may acquire the bed departure or the bed presence of the user based on the amount of activity.

An RASS evaluation unit 140 determines RASS serving as a sedative scale of a user. The RASS evaluation unit 140 can determine at least to which RASS group the RASS of a user belongs based on a threshold stored in a determination threshold table 206, for example. The processing by the RASS evaluation unit 140 is described later. The RASS group herein indicates that RASS scales are classified into a plurality of groups.

A disease information acquisition unit 150 acquires disease information that is information related to a disease of a user. The disease information acquisition unit 150 may be connected to an electronic medical record server, for example, and acquire disease information on the user. The disease information acquisition unit 150 may refer to information input by a medical doctor or the like, and acquire disease information. In the present embodiment, the disease information acquisition unit 150 may acquire, as disease information, a type and an amount of a drug, such as an anesthetic drug and a sedative drug, administered to the user.

The storage 210 stores biological information data 202, user state data 204, and the determination threshold table 206, and secures an area of a parameter buffer area 208.

The biological information data 202 stores, for example, an amount of activity, as information related to the biological information value calculated from the acquired biosignal (body movement) by the controller 100. In the present embodiment, as information related to the biological information value, the amount of activity is stored, and a respiratory rate, a heartbeat rate, and the like may further be stored. The biological information data 202 preferably stores information related to the biological information value in a time-series manner for every predetermined time.

The user state data 204 stores a state of a user. As a state of a user acquired by the user state acquisition unit 130, whether the user is in a “bed presence” or “bed departure” state is stored. The user state data 204 may further store the state of the user by including the sleeping state determined by the sleeping state determination unit 120. For example, if the user state acquisition unit 130 has determined that the user is in a “bed presence” state, the sleeping state determined by the sleeping state determination unit 120 may be stored. The user state data 204 may preferably stores states of a user in a time-series manner for every predetermined time.

The determination threshold table 206 is a table in which thresholds to be used when the controller 100 (RASS evaluation unit) determines RASS of a patient are stored. FIG. 3B illustrates one example of the determination threshold table 206.

In the determination threshold table, a first determination threshold (for example, “47”) and a second determination threshold (for example, “66”) are stored.

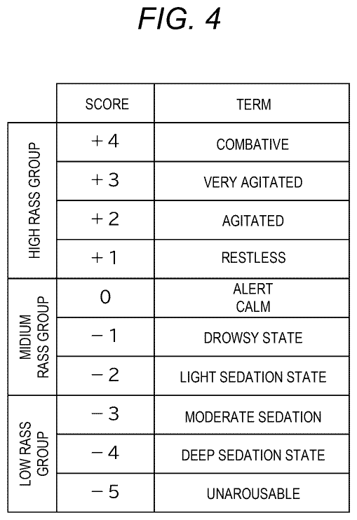

In the present embodiment, thresholds of the amount of activity with which the RASS of the patient is classified into a low RASS group, a medium RASS group, or a high RASS group are stored. A relation between the RASS groups and the RASS will be described herein with reference to FIG. 4. The RASS is normally classified into scores of “+4” to “−5”. The high RASS group indicates, for example, the scores of RASS of “+4” to “+1”. The medium RASS group indicates, for example, the scores of RASS of “0” to “−2”. The low RASS group indicates, for example, the scores of RASS of “−3” to “−5”.

The scores of RASS and terms in FIG. 4 are merely examples, and based on Guidelines for sedation practicing artificial respiration, Japanese Society of Intensive Care Medicine, (2007). Other definitions may be used for the classification method of the scores of RASS and the meaning of terms. For example, in a case where RASS is applied to RASS for palliative care, the score “−5” may be used as a term of “unable to wake”.

The amount of activity less than the first determination threshold is set to the low RASS group in the present embodiment. The amount of activity equal to or more than the second determination threshold is set to the high RASS group in the present embodiment. The amount of activity equal to or more than the first determination threshold and less than the second determination threshold is set to the medium RASS group.

In the present embodiment, RASS scales are classified into three groups. This is the most effective way of classification in a case of being based on the example to be described later, but RASS scales may be classified into two or four groups. The RASS scales may be identical with the RASS groups.

### 1.3 Flow of Processing

RASS evaluation processing in the present embodiment will be described. A description will be made in the description of the processing by assuming that processing that is executed as appropriate by the functional unit (for example, the RASS evaluation unit 140) illustrated in FIG. 3 is to be executed by the controller 100, for convenience of the description.

### 1.3.1 Overall Flow

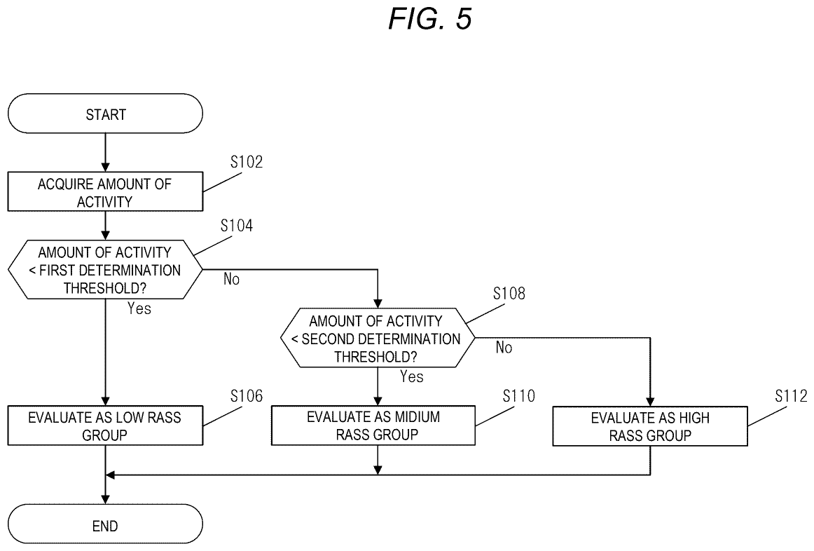

FIG. 5 is an operation flow illustrating the RASS evaluation processing in the present embodiment. The controller 100 acquires an amount of activity (S102). For example, the controller 100 may calculate an amount of activity in predetermined time (for example, for every second) by the biological information value calculation unit 110. The controller 100 may calculate an amount of activity, for example, at intervals, such as for every 5 seconds and for every 10 seconds, as timing of the predetermined time. The controller 100 may calculate an amount of activity at timing (for example, 100 milliseconds or the like) shorter than one second.

If the controller 100 has determined that the amount of activity is less than the first determination threshold (S104; Yes), the controller 100 evaluates the patient as the low RASS group (S106). The first threshold herein is, for example, “47”.

If the controller 100 has determined that the amount of activity is less than the second determination threshold (S108; Yes), the controller 100 evaluates the patient as the medium RASS group (S108; Yes→S110). In other words, if the amount of activity is equal to or more than the first determination threshold and less than the second determination threshold, the controller 100 evaluates the patient as the medium RASS group.

In the other cases, that is, if the amount of activity is equal to or more than the second determination threshold, the controller 100 evaluates the patient as the high RASS group.

In the present embodiment, the controller 100 compares the amount of activity with the determination thresholds to result in the evaluation of the low RASS group, the medium RASS group, and the high RASS group, but several methods can be considered as the method of evaluation.

For example, the controller 100 specifies a median value from the measured amount of activity, and divides the amount of activity into the RASS groups based on the ratio using the median value as the center. For example, the controller 100 sets a median value of the amount of activity as the center, and sets 30% of the entire values including the median value as a medium RASS group. The controller 100 then specifies a group of values lower than those in the medium RASS group as a low RASS group, and a group of values higher than those in the medium RASS group as a high RASS group.

The controller 100 may perform grouping by simply using a standard deviation (σ). For example, the controller 100 uses an amount of activity in an individual determined in the past specific period (for example, for past seven days) to evaluate whether the current amount of activity is high (high RASS group) or low (low RASS group) for the individual. For example, if the measured amount of activity is a value exceeding 2σ that is the amount of activity in the specific period, the controller 100 evaluates the value as the low RASS group or the high RASS group.

When RASSs are divided into these groups, the controller 100 may change the order of the measured RASSs among a plurality of persons not in an individual in descending order of the amounts of activity, and evaluates the persons in descending order of the amounts of activity.

The controller 100 may use a trained model to evaluate the low RASS group, the medium RASS group, and the high RASS group. For example, a trained model is generated using the amount of activity of a patient and multiple parameters (for example, biological information, medication information) as explanatory variables, and a score of RASS of the patient determined by a staff or the like as labeled data. The controller 100 can obtain an RASS score of a patient by putting at least the amount of activity into the trained model. This allows the controller 100 to evaluate the RASS score as the low RASS group, the medium RASS group, or the high RASS group by using the trained model.

### 1.4 Example

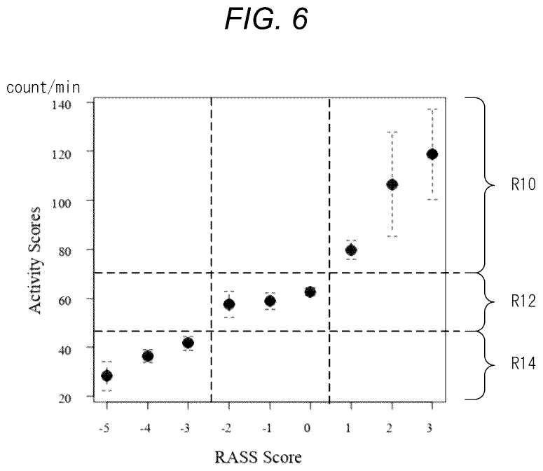

As an example, the relation between the amount of activity and the score of RASS is illustrated as a graph in FIG. 6. The graph in FIG. 6 illustrated as the example is a graph based on the average value when measurement using RASS for palliative care was conducted with respect to 971 patients (551 male patients, 420 female patients) in the terminal phase of cancer at the average age of 74.2. When the average value is plotted, characteristics are indicated respectively as R10 for groups of the high RASS group (RASS score is equal to or more than 1), as R12 for groups of the medium RASS group (the RASS score is from −2 to 0), and R14 for groups of the low RASS group (the RASS score is equal to or less than −3).

In other words, the controller 100 can evaluate, based on the amount of activity of a patient, whether the RASS score of the patient is included in the high RASS group, the medium RASS group, or the low RASS group.

### 1.5 Effects

In this manner, with the present embodiment, it is possible to easily evaluate the RASS score of a patient based on the amount of activity. The RASS score of a patient has been evaluated from a subjective point of view by an evaluator, but can be objectively evaluated by using the amount of activity.

The amount of activity of a patient can be acquired all the time by using the measuring device, so that the RASS score can be continuously evaluated. Accordingly, for example, as for a symptom and the like of delirium that a staff or the like easily fails to notice in normal clinical practice, by continuously monitoring the amount of activity all the time, it is possible to detect an increase (or a decrease) in the activity indicating developing of restlessness (or oversedation), which is a symptom of hyperactive type delirium.

The restlessness, which is a symptom of delirium in a patient in the terminal phase, and the sedative level can be quantitated, so that it is possible to provide a proper indicator to care for the patient.

### 2. SECOND EMBODIMENT

A second embodiment will be described. The second embodiment is an embodiment in which by using the RASS evaluation in the first embodiment, treatment for a patient and coping related to the medication can be displayed, for example.

Firstly, when a patient is selected, the controller 100 executes the RASS evaluation processing described in the first embodiment, and evaluates the RASS score (or RASS group) of the patient (S202→S204).

The controller 100 then acquires treatment information that is information related to treatment made to the patient (S206). The controller 100 only needs to acquire the treatment information from any source, and may acquire the treatment information by making an inquiry to an electronic medical record server, for example. For example, the disease information acquisition unit 150 acquires treatment information. The treatment information includes, for example, information on a drug (sleep drug, sedative drug) administered to the patient.

The controller 100 outputs a coping content based on the acquired treatment information and the RASS evaluation (S208). The controller 100 then issues a doctor call if having determined that the doctor call is necessary as needed (S210; Yes→S212). The controller 100 then causes the processing to return to S204 and repeatedly executes the processing, thereby monitoring the state of the patient all the time.

Here, the coping content that is output by the controller 100 will be described in details. For example, the memory 200 may include information (coping information) related to the coping in accordance with the administered drug. For example, as one example, a case where Midazolam, which is a benzothiazepine anesthetic induction drug and a sedative drug, and Propofol, which is an agent for general anesthesia and sedation are used in an intensive care unit (ICU) will be described hereinafter.

For example, the controller 100 displays that the administration is reduced by 1 mL/hr when the RASS score of a patient was evaluated as from “−5” to “−3”. The controller displays that the current state may be maintained when the RASS score of a patient was evaluated as from “−2” to “0”. The controller 100 displays that the administration is reduced by 1 mL/hr when the same RASS score continues in two hours.

The controller 100 displays that 2 mL of the drug is bolus-administered when the RASS score of a patient was evaluated as from “+1” to “+3”. The controller 100 may display that the administration is made twice at intervals of 15 minutes. When the RASS score does not change after the administration twice, the controller 100 displays that the administration is increased by 1 mL/hr if the patient is under the continuous administration. The controller 100 displays that the administration is restarted at the initial dosage if the medication to the patient is being suspended.

The controller 100 displays that 2 mL of the drug is bolus-administered when the RASS score is “+4”. In addition, the controller 100 determines that a doctor call needs to be issued, and issues the doctor call.

In a case of other drugs, the controller 100 may issue a doctor call even when the RASS score of a patient was evaluated as from “+1” to “+3”. For example, in a case of Dexmedetomidine, which is an α2 agonistic sedative drug, when the RASS score was evaluated as from “+1” to “+3”, the controller 100 issues a doctor call if the medication to the patient is being suspended.

In this manner, with the present embodiment, in accordance with the RASS score of a patient, the controller 100 can output the coping content or issue a doctor call.

### 3. THIRD EMBODIMENT

A third embodiment will be described. The third embodiment is an embodiment in which the RASS score or the RASS group of a patient is stored, and displayed or printed, for example. The controller 100 stores the RASS score or the RASS group of a user on a time-series basis in the memory 200.

The controller 100 displays the RASS scores and RASS groups stored on a time-series basis to allow a staff or the like to easily grasp a past state of the user, for example.

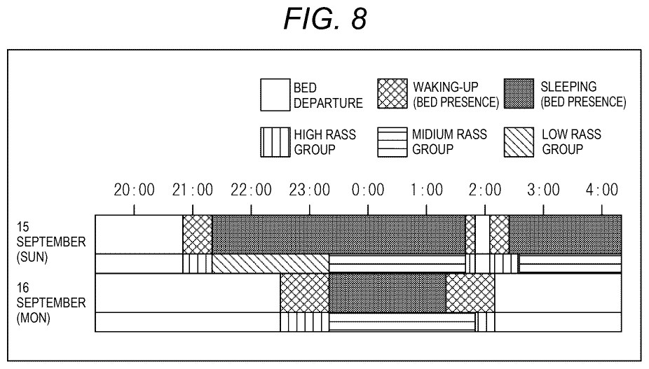

In this process, the controller 100 may display, for example, other information together with the RASS score. For example, FIG. 8 illustrates one example of a sleep diary that is displayed in the third embodiment. The sleep diary displays a sleeping state of a patient, for example, bed departure and bed presence (waking-up or sleeping) for every date are graph displayed as a component bar chart.

In the present embodiment, the controller 100 displays information related to RASS together with the sleeping state. For example, FIG. 8 displays a sleeping state in the upper part and the RASS group in the lower part, respectively, for every date. In this manner, the controller 100 can display a plurality of past states (a biological information value, a state of sleep, a score of RASS, and a RASS group) related to the patient. The information related to RASS is displayed together with the conventional biological information value and the state of sleeping to allow a staff or the like to administer suitable medication and make the rounds.

### 4. FOURTH EMBODIMENT

A fourth embodiment is an embodiment in a case where a determination threshold is changed. For example, the controller 100 can change the first determination threshold and the second determination threshold.

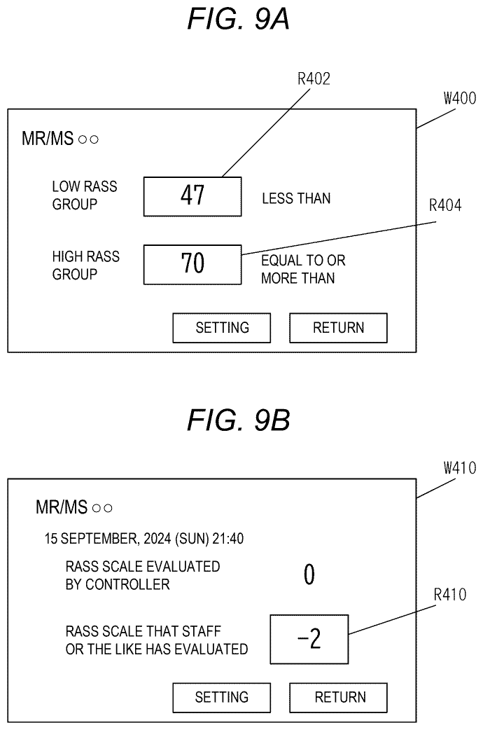

FIG. 9A is a diagram illustrating one example of a display screen W400 in a case where a staff or the like changes a determination threshold to any numerical value. For example, the display screen W400 includes a region R402 in which a first determination threshold can be input, and a region R404 in which a second determination threshold can be input. The controller 100 stores values input into the regions R402 and R404 as a first determination threshold and a second determination threshold, respectively. A first determination threshold and a second determination threshold may be set for every patient. In this case, the determination thresholds are stored in association with a patient ID or the like, for example.

The controller 100 may automatically set the determination threshold by using machine learning. In other words, a trained model that outputs the RASS score of a patient may further be stored in the memory 200.

For example, as an explanatory variable, a biological information value (amount of activity) of a patient is input to a trained model, and as a response variable, an RASS score is output from a trained model. As explanatory variables, at least information (for example, information related to an age, a gender, and a disease) on a patient, and an amount of activity are input. As explanatory variables, biological information values (heartbeat rate, respiratory rate) of a patient, and disease information (medication information) may further be input. A staff or the like then evaluates an RASS score of the state of the patient, and inputs the RASS score as labeled data. The controller 100 performs machine learning by using these parameters to generate a trained model that outputs the RASS score as a response variable, and performs relearning.

FIG. 9B illustrates one example of a display screen W410 in which a staff or the like inputs a new RASS scale when the controller 100 relearns a trained model. For example, an RASS scale of a patient currently evaluated by the controller 100 is displayed on the display screen W410.

A region R410 indicates an RASS scale that the staff or the like has evaluated. This allows the trained model to be relearned based on the new RASS scale provided by the staff or the like.

The trained model may be applied for each patient, or may be applied for each facility. Data may be collectively collected on a service provider side. The trained model is adjusted based on much data on the service provider side to allow a more accurate trained model to be generated.

### 5. FIFTH EMBODIMENT

A fifth embodiment is an embodiment in which the controller 100 is in cooperation with another device by using the RASS scale evaluated in the first embodiment.

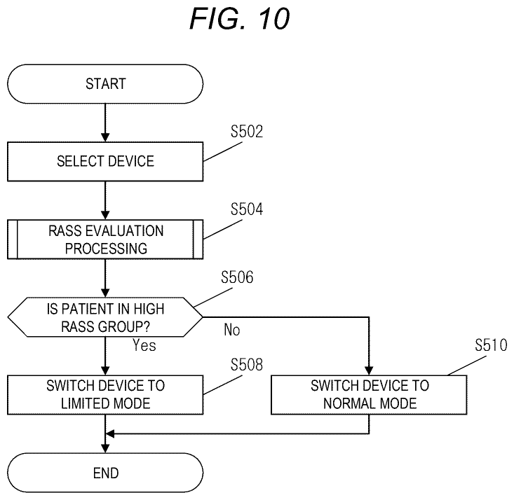

An operation in the fifth embodiment will be described with reference to FIG. 10. The controller 100 firstly selects a device to be controlled (S502). The controller 100 then executes the RASS evaluation processing in the first embodiment, and evaluates an RASS scale or an RASS scale group of a patient (S504).

As one example, the controller 100 evaluates an RASS scale group of the patient, and switches the device to a limited mode if the RASS scale group is evaluated as a high RASS group (S508). On the other hand, if the RASS scale group of the patient is other than the high RASS group, the controller 100 switches the device to a normal mode. The following examples can be considered as the device to be selected.

### (1) Bed 3

Operation modes in a back-raising operation, a knee-raising operation, and the like in the bed 3. For example, if the controller 100 has evaluated that the patient is included in the high RASS group, the controller 100 may set a back-raising angle to a more limited range than usual (for example, the range allowing the back-raising operation is limited to 0 degrees to 20 degrees, whereas the back-raising operation is usually allowed from 0 degrees to 75 degrees).

The controller 100 may deform sections of the bed 3 in a flat state in the limited mode.

The controller 100 may control the height of the bed 3. For example, the controller 100 may control the height of the bed 3 in the limited mode to be lower than that in the normal mode. For example, the controller 100 can perform an operation of lowering the height of the bed 3 if the RASS scale group of the patient has been evaluated as the high RASS group. The controller 100 may perform the operation of lowering the height of the bed 3 also based on the amount of activity of the patient.

### (2) Change in Threshold of Bed Departure Sensor

For example, the controller 100 may change a threshold at which a sensor (bed departure sensor) capable of detecting bed departure of a patient determines the bed departure. For example, the controller 100 may set a low threshold in the limited mode, and report a possibility of bed departure from a slight motion by a patient.

### (3) Range of Abnormal Value Report of Vital Sensor

For example, the controller 100 can output an alarm or call a staff when a biological information value of a patient has exceeded (fallen below) a predetermined threshold set in advance. The controller 100 normally makes a report by outputting an alarm (for example, displaying a warning on the screen or outputting an alarm sound), but in a case of the limited mode, the controller 100 may make a push notification with an email to the staff or an application, for example.

In this manner, with the present embodiment, it is possible to appropriately control another device based on the state (RASS) of the patient.

### 6. MODIFIED EXAMPLES

The present disclosure is not limited to each of the above-described embodiments, and various modifications can be made thereto.

That is, embodiments that can be implemented by combining technical means appropriately modified within the scope that does not depart from the gist of the present disclosure are also included in the technical scope. The above-described respective embodiments applied to the RASS for palliative care have been described as preferred examples, but may be applied to the RASS and the like of an ICU patient.

The above-described embodiments are divided for convenience of the description. However, the embodiments can be combined and implemented within the realm of possibility. It is also intended to acquire the rights to any of the techniques disclosed in the present disclosure in the forms of an amendment, a divisional application, and the like.

Moreover, the program that operates in each device in the respective embodiments is a program that controls a CPU or the like (a program that causes a computer to function) so as to implement the functions in the abovementioned embodiments. Further, the information that is handled by these devices is temporarily stored in a temporary storage (for example, RAM) at the time of processing, and thereafter stored in various ROM and HDD storage devices, and is read and corrected/written by the CPU as necessary.

Herein, as a recording medium that stores the program, any of a semiconductor medium (for example, a ROM and a nonvolatile memory card), an optical recording medium, a magneto-optical recording medium (for example, a digital versatile disc (DVD), a compact disc (CD), and a Blu-ray (registered trademark) disc (BD)), a magnetic recording medium (for example, a magnetic tape and a flexible disc), and the like may be used.

Further, in a case of distribution to the market, the program can be stored and distributed in a portable recording medium, or transferred to a server computer connected via a network such as the Internet. In this case, a storage device of the server device, of course, falls within the present disclosure.

The above-described data is not stored in the device, but may be stored in an external device, and called, as appropriate. For example, data may be stored in a network attached storage (NAS), or stored on a cloud.

The scope of the present disclosure is not limited to the configurations that are explicitly described in the present disclosure, and includes combinations of the techniques disclosed in the present disclosure. In the present disclosure, the configurations, for which the applicant attempts to acquire a patent, are described in the claims. However, it is not intended that the configuration not described in the claims is excluded from the technical scope.

In the above-described specification, each of the expressions such as “in the case where . . . ” and “when . . . ” only represents one example, and thus the present disclosure is not limited to the contents described above. Configurations not described with these expressions are also disclosed within the scope that is apparent for those skilled in the art, and it is intended to acquire the rights to such configurations.

Orders of the processing and the data flow described in the present specification are not limited to the described orders. For example, the configuration in which the processing is partially omitted and the configuration in which the order of the processing is changed are also disclosed, and it is intended to acquire the right therefor.

In the description, the functions described in the embodiments are executed in the respective devices, but may be implemented in one device, and an external server may further be used.

The respective functional blocks or various features of the devices used in the above-described embodiments can be implemented or executed by an electric circuit, for example, an integrated circuit or a plurality of integrated circuits. Electrical circuits designed to execute the functions described in the present specification may include general purpose processors, digital signal processors (DSPs), application specific integrated circuits (ASICs), field programmable gate arrays (FPGAs), or other programmable logic devices, discrete gate or transistor logic, discrete hardware components, or combinations thereof. The general purpose processor may be a microprocessor or a processor, a controller, a microcontroller, or a state machine of the related art. The above-described electric circuit may be implemented by a digital circuit or may be implemented by an analog circuit. In a case where a technology of an integrated circuit that replaces the current integrated circuit appears due to the progress of the semiconductor technology, one or more aspects in the present disclosure can also use a new integrated circuit based on the technology.

The processor 14 outputs biological information based on a result output from the detection device 12 in the present embodiment, however, the detection device 12 may calculate the whole. An application is installed and implemented not only in a terminal device (for example, a smartphone, a tablet, and a computer), but also processing may be performed at a server side and a process result may be returned to the terminal device, for example.

For example, the detection device 12 may upload biological information to the server, thereby implementing the above-described processing at the server side. The detection device 12 may be implemented by a device such as a smartphone in which an acceleration sensor and a vibration sensor are incorporated therein, for example.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An evaluation apparatus comprising:
a biosignal acquisition unit configured to acquire a biosignal of a patient; and
a controller, wherein
the controller is configured to
calculate an amount of activity of the patient based on the biosignal acquired from the biosignal acquisition unit, and
evaluate a sedative scale (Richmond Agitation-Sedation Scale (RASS)) of the patient based on the calculated amount of activity.

2. The evaluation apparatus according to claim 1, wherein the controller compares the amount of activity with a determination threshold to evaluate the sedative scale of the patient as a low RASS group, a medium RASS group, and a high RASS group.

3. The evaluation apparatus according to claim 1, wherein the controller outputs a coping content against the patient based on the sedative scale.

4. The evaluation apparatus according to claim 1, wherein
the controller acquires a sleeping state of the patient, and
outputs a sleep diary including the sleeping state of the patient, and the sedative scale.

5. An evaluation system comprising:
a sheet-shaped detection device that is placed under a patient, the detection device being configured to detect a body movement of the patient;
a calculation device configured to calculate an amount of activity of the patient from the body movement; and
an evaluation apparatus configured to evaluate a sedative scale (Richmond Agitation-Sedation Scale (RASS)) of the patient based on the amount of activity.
